(19) United States
(12) Patent Application Publication
(10) Pub. No.: US 2025/0339602 A1
(43) Pub. Date: Nov. 6, 2025

(54) AN AIRWAY FOR A WOUND THERAPY DEVICE

(71) Applicant: ConvaTec Limited, Flintshire (GB)

(72) Inventor: Chandan Rangaiah, Warrington (GB)

(21) Appl. No.: 19/265,261

(22) Filed: Jul. 10, 2025

Related U.S. Application Data
(63) Continuation of application No. PCT/GB2024/050055, filed on Jan. 10, 2024.
(60) Provisional application No. 63/438,883, filed on Jan. 13, 2023.

(30) Foreign Application Priority Data
Feb. 22, 2023 (GB) 2302558.8

(51) Int. Cl. A61M 1/00; A61F 13/05

(52) U.S. Cl. CPC A61M 1/94; A61F 13/05; A61M 1/92; A61M 2205/02

(57) ABSTRACT

An airway for a pressure gradient wound therapy device comprising a transmission layer comprising a proximal end, an elongate middle portion and a distal end, the transmission layer comprising an upper layer and a lower layer defining a space therebetween; a top layer constructed from a liquid impermeable material provided over the transmission layer; a bottom layer constructed from a liquid impermeable material provided below the transmission layer, wherein the top layer and the bottom layer enclose at least a portion of the transmission layer; and a conduit comprising a proximal end and a distal end, wherein the proximal end of the conduit is arranged in the space between the upper and lower layers of the proximal end of the transmission layer.

### TECHNICAL FIELD OF THE INVENTION

The present invention relates to an airway. In particular, the invention concerns an airway for a pressure gradient wound therapy device, and most particularly, but not exclusively, an airway for a negative pressure wound dressing.

### BACKGROUND TO THE INVENTION

Wound dressings are known and are generally suitable for treating a variety of wounds, including chronic and acute wound types, such as infected wounds, venous ulcers, diabetic ulcers, burns and surgical wounds.

Negative pressure has been used to treat a range of chronic and acute wounds. Negative pressure may facilitate wound healing through a number of mechanisms, including removal of excess exudate, reduction in periwound edema and increased perfusion. Combined with the physical forces exerted by the negative pressure which draw the wound edges together, this can result in improved wound outcomes. Existing wound dressing systems typically rely on an airway having a conduit to draw air away from the wound site via a pump.

One exemplary known airway is attached to Pico (RTM) dressings sold by Smith and Nephew (RTM). This airway is a generally flat arrangement with a top layer, and a bottom layer which together define a channel, the channel with a spacer layer enclosed between the top and bottom layers. Typically, the top and bottom layers are each a layer of flexible, transparent, liquid impermeable film, and are often formed of polyurethane. The spacer layer serves to keep the film layers separate to ensure that there is an open path for fluid to travel between the wound dressing and a source of negative pressure. For connection to a source of negative pressure, a short piece of transparent tubing extends from the end of the airway opposite to the wound dressing.

The spacer layer comprises an elongate portion (extending substantially the length of the channel) and a folded end which surrounds a first end of the tubing. The folded end is folded upward, toward the top layer, then toward itself so that the folded end is substantially parallel with the elongate portion. The fold comprises an aperture through which a first end of the conduit is inserted. The first end of the conduit is positioned between the folded portion of the spacer fabric and the elongate portion of the spacer fabric. Thus, the spacer layer maintains an open path for fluid from the wound dressing to the tubing. With a layer of spacer fabric on top and below, the film layer cannot close off the open first end of the tubing.

The arrangement of the prior art seemingly aims to reduce the possibility of an end of the conduit piercing either layer of film. However, the arrangement is flawed in that the conduit can easily be inserted too far through the aperture in the fold and extend beyond the folded portion of spacer fabric. This creates a pressure point between the conduit and the top film layer of which the conduit is then prone to piercing the top film layer. In this event, the patient may suffer from discomfort should the conduit protrude into the patient's skin. However, this discomfort can also occur when the conduit extends beyond the folded portion of the spacer fabric and does not pierce the top film layer. Further, in the event that the conduit pierces the top film layer, the conduit would no longer be able to transmit a negative pressure to the wound site, therefore, adversely affecting healing and recovery of the wound.

Moreover, if the conduit is inserted too far through the aperture in the fold and extends beyond the folded portion of spacer fabric then, in use, the draw of air away from a wound site into the conduit would cause the top film layer to be drawn into the conduit, covering the conduit and, therefore, preventing the conduit transmitting a negative pressure to a wound site.

The arrangement of the prior art described above provides an airway which is relatively thin compared to, for example, wound dressings having solid parts (e.g., hard plastic tubing) extending from a backing layer of the dressing.

It is an object of embodiments of the present invention to at least partially overcome or alleviate the above problems and/or to provide an improved airway for a wound dressing.

### SUMMARY OF THE INVENTION

In broad terms, the present invention concerns an airway for a pressure gradient wound therapy device.

According to a broad aspect of the present invention, there is provided an airway for a pressure gradient wound therapy device comprising a transmission layer and a conduit:
the transmission layer comprising an upper layer and a lower layer and defining a space therebetween; and
the conduit comprising a proximal end and a distal end, wherein the proximal end of the conduit is arranged in the space between the upper and lower layers of the transmission layer.

The transmission layer may comprise a proximal end. The transmission layer may comprise a distal end. The transmission layer may comprise a middle portion (which may be arranged between the proximal end and the distal end). The middle portion may be elongate. The airway may comprise a top layer, which may be constructed from a liquid impermeable material and may be provided over the transmission layer. The airway may comprise a bottom layer, which may be constructed from a liquid impermeable material and may be provided below the transmission layer. The top layer and the bottom layer may enclose (or at least substantially enclose) at least a portion of the transmission layer. The proximal end of the conduit may be arranged in the space between the upper and lower layers of the proximal end of the transmission layer.

As such, a first aspect of the invention provides an airway for a pressure gradient wound therapy device comprising: a transmission layer comprising a proximal end, an elongate middle portion and a distal end, the transmission layer comprising an upper layer and a lower layer defining a space therebetween; a top layer constructed from a liquid impermeable material provided over the transmission layer; a bottom layer constructed from a liquid impermeable material provided below the transmission layer, wherein the top layer and the bottom layer enclose at least a portion of the transmission layer; and a conduit comprising a proximal end and a distal end, wherein the proximal end of the conduit is arranged in the space between the upper and lower layers of the proximal end of the transmission layer.

Advantageously, the present invention provides an airway which can be of reduced thickness compared to airways of the prior art. Since the conduit is arranged within the transmission layer (i.e., in the space between the upper and lower layers of the transmission layer), the transmission layer is not required to be folded and as such, the thickness of the airway is reduced by a distance approximately equal to the thickness of the transmission layer. This is because instead of having two layers of transmission layer in the folded region, one on top of the conduit and one below it, there is only one layer of transmission layer, with the conduit provided inside it. As such, the present invention provides an airway which can be more comfortable for a patient should the airway be rested or lent upon by the patient. Moreover, the airway is therefore less likely to cause pressure ulcers and other complications which could otherwise result in significant distress and pain for the patient.

Further advantageously, the airway of the present invention prevents, or at least significantly reduces the probability of, the conduit piercing a film layer of the airway. This is because the proximal end of the conduit is arranged in the space between the upper and lower layers of the transmission layer. As such, the upper and lower layers of the transmission layer act as a physical barrier between the proximal end of the conduit and the film layers positioned above and below the transmission layer to prevent, or at least substantially reduce the probability of, the conduit piercing the film layers. Since the conduit is within the transmission layer, in the event that it is pushed further into the layer, as described in relation to the folded arrangement of the prior art, it will not extend out and pierce a film. Thus, the present invention provides an airway which is more reliable in transmitting a negative pressure to a wound site compared to airways of the prior art.

Moreover, the present invention is far simpler to manufacture compared to airways of the prior art. The above-described airway of the prior art requires careful insertion of a conduit into an aperture through the plane of the spacer layer and the creation of a fold in the spacer layer, at the aperture, such that the conduit is arranged within the folded part. As previously stated, if the conduit is inserted too far through the folded part, the conduit is prone to piercing the film of the airway, therefore, careful preparation of the airway of the prior art is critical. Comparatively, the present invention is simple to manufacture in that all that is required is the proximal end of the conduit to be arranged in the space between the upper and lower layers of the transmission layer. As such, the distance that the conduit is inserted is much less critical, so the present invention provides an airway which is easy to manufacture, and which offers little to no risk of damaging the components of the airway.

The top layer may be formed from a material which is gas impermeable. The top layer may be formed from a material that is moisture vapour permeable. The top layer may be a polyurethane film.

The bottom layer may be formed from a material which is gas impermeable. The bottom layer may be formed from a material which is moisture vapour permeable. The bottom layer may be a polyurethane film.

Advantageously, a liquid and gas impermeable, but moisture vapour permeable top and/or bottom layer ensures that gas does not escape from the airway, reducing the vacuum; that liquid does not escape from the airway (e.g. on route to a cannister, if the NPWT system is one including a cannister), but allows moisture vapour to escape, reducing the quantity of moisture that must be held in the cannister.

The top layer and the bottom layer may be formed of the same material.

The top layer and the bottom layer may each be formed of polyurethane, thermoplastic polyurethane, thermoplastic elastomer, polyethylene, polypropylene, polyvinyl chloride, ethylene-vinyl acetate, polyester or a silicone material.

The top layer and the bottom layer may enclose, the proximal end of the transmission layer. The top layer and the bottom layer may enclose the proximal end and the elongate middle portion of the transmission layer. The top layer may enclose the proximal end, elongate middle portion and the distal end of the transmission layer and the bottom layer may enclose the proximal end and at least part, for example a major part, of the elongate middle portion. The bottom layer may not enclose the distal end of the transmission layer. An aperture may be provided in the bottom layer for connection to an opening in a wound dressing.

The proximal end of the conduit may be adhered to the transmission layer within the space between the upper and lower layers of the transmission layer. The proximal end of the conduit may be adhered to one or more inner surfaces of the proximal end of the transmission layer.

The proximal end of the conduit may be adhered to the transmission layer by an adhesive, a weld, such as a heat weld, heat lamination, pressure lamination, or heat and pressure lamination. Advantageously, this means that the conduit is firmly secured within the transmission layer, therefore, providing a more reliable airway for use with a negative pressure wound dressing. Alternatively, the top and bottom layers may secure the conduit and the transmission layer in position.

The proximal end of the conduit may be adhered to the transmission layer around an aperture in the proximal end of the transmission layer through which the proximal end of the conduit is inserted so that it is arranged in the space between the upper layer and lower layer of the transmission layer.

The conduit may be formed of polyurethane, thermoplastic polyurethane, thermoplastic elastomer, polyethylene, polypropylene, polyvinyl chloride, ethylene-vinyl acetate, polyester or a silicone material. For example, the conduit may be medical tubing. The conduit allows the passage of fluid (i.e., a gas or a liquid) along its length. The distal end of the conduit may be provided with a connector for connection to a source of negative pressure. For example, the distal end may be provided with a luer-lock connector.

The conduit may have an external diameter of at least about 1 mm, about 2 mm, about 3 mm, about 4 mm, about 5 mm, about 6 mm, about 7 mm, about 8 mm, about 9 mm, or at least about 10 mm

The conduit may have an external diameter of no more than about 1 mm, about 2 mm, about 3 mm, about 4 mm, about 5 mm, about 6 mm, about 7 mm, about 8 mm, about 9 mm, or no more than about 10 mm

The conduit may have an external diameter of about 1 mm, about 2 mm, about 3 mm, about 4 mm, about 5 mm, about 6 mm, about 7 mm, about 8 mm, about 9 mm, or about 10 mm.

The conduit may have an internal diameter of at least about 0.5 mm, about 1.0 mm, about 1.5 mm, about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8.0 mm, about 8.5 mm, about 9.0 mm, or at least about 9.5 mm.

The conduit may have an internal diameter of no more than about 0.5 mm, about 1.0 mm, about 1.5 mm, about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8.0 mm, about 8.5 mm, about 9.0 mm, or no more than about 9.5 mm.

The conduit may have an internal diameter of about 0.5 mm, about 1.0 mm, about 1.5 mm, about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8.0 mm, about 8.5 mm, about 9.0 mm, or about 9.5 mm.

The transmission layer may be a polyurethane foam. The transmission layer may be a nylon knitted mesh. The transmission layer may be a 3D spacer fabric. The transmission layer may be a 3D knitted material. The transmission layer may be a foam, for example a reticulated foam. The transmission layer may be a porous material. The transmission layer may be a non-woven material. Advantageously, each of these materials are cheap and readily available. Moreover, many of these materials are recyclable and reusable, and can be formed from recycled materials.

The transmission layer may be a foam, for example a polyurethane foam.

The transmission layer may be a knitted material, for example a knitted mesh, for example a nylon knitted mesh. The knitted material may be a 3D knitted material.

The transmission layer may be a spacer fabric, for example a 3D spacer fabric. The spacer fabric may be a polyester spacer fabric. The spacer fabric may comprise a microfilament yarn between two fabric layers. The yarn and/or one or each of the fabric layers may be formed of polyester.

The transmission layer may be a porous material.

The transmission layer may be a non-woven material.

Advantageously, each of these materials are cheap and readily available. Moreover, many of these materials are recyclable and reusable, and can be formed from recycled materials.

In an embodiment of the invention, there is provided an airway for a pressure gradient wound therapy device comprising: a transmission layer comprising a proximal end, an elongate middle portion and a distal end, the transmission layer comprising an upper layer and a lower layer defining a space therebetween; a top layer constructed from a liquid impermeable material provided over the transmission layer; a bottom layer constructed from a liquid impermeable material provided below the transmission layer, wherein the top layer and the bottom layer enclose at least a portion of the transmission layer; and a conduit comprising a proximal end and a distal end, wherein the proximal end of the conduit is arranged in the space between the upper and lower layers of the proximal end of the transmission layer, wherein the transmission layer is a 3D spacer fabric, a 3D knitted material or a non-woven material.

The transmission layer is preferably formed of a material comprising a plurality of monofilaments separating the upper and lower layers of the transmission layer. In particular, the transmission layer may be formed of a 3D spacer fabric, a 3D knitted material or a non-woven material comprising a plurality of monofilaments separating the upper and lower layers. The proximal end of the conduit may be adhered to one or more of the monofilaments adjacent to the proximal end of the conduit in the space. This adhesion may be in addition to an adhesion between the proximal end of the conduit and an inner surface of the upper and lower layers of the transmission layer.

The upper layer and the lower layer of the transmission layer may be joined to one another by the plurality of monofilaments separating the upper and lower layers of the transmission layer.

The upper layer and the lower layer of the transmission layer may be joined to one another about their periphery. For example, the upper layer and the lower layer may each comprise a peripheral edge, and the peripheral edge of the upper layer may be joined to the peripheral edge of the lower layer. The join may be formed by stitching, an adhesive, or welding, such as a heat-weld or a pressure weld.

The space between the upper layer and the lower layer of the transmission layer may be enclosed within the confines of the transmission layer. In embodiments wherein the upper layer and the lower layer of the transmission layer are joined to one another about their periphery, the space may be enclosed within the confines of the upper layer, lower layer and the periphery of the joined upper and lower layers.

The 3D spacer fabric may be formed of polyester. The 3D spacer fabric may comprise filaments and yarn each formed of polyester.

The transmission layer may be formed by warp knitting, weft knitting or circular knitting.

In an embodiment of the invention, there is provided an airway for a pressure gradient wound therapy device comprising: a transmission layer comprising a proximal end, an elongate middle portion and a distal end, the transmission layer comprising an upper layer and a lower layer defining a space therebetween; a top layer constructed from a liquid impermeable material provided over the transmission layer; a bottom layer constructed from a liquid impermeable material provided below the transmission layer, wherein the top layer and the bottom layer enclose at least a portion of the transmission layer; and a conduit comprising a proximal end and a distal end, wherein the proximal end of the conduit is arranged in the space between the upper and lower layers of the proximal end of the transmission layer, wherein the transmission layer is a 3D spacer fabric comprising a plurality of monofilaments separating the upper and lower layers of the transmission layer.

The transmission layer may have a basis weight of between about 100 and about 500 g/m², about 110 and about 475 g/m², about 120 and about 450 g/m², about 130 and about 425 g/m², about 140 and about 400 g/m², about 150 and about 375 g/m², about 160 and about 350 g/m², about 170 and about 325 g/m², about 180 and about 300 g/m², about 190 and about 275 g/m², about 200 and about 250 g/m², about 210 and about 240 g/m², about 220 and about 230 g/m², or about 225 g/m².

The transmission layer may have a basis weight of about 100 g/m², about 110 g/m², about 120 g/m², about 130 g/m², about 140 g/m², about 150 g/m², about 160 g/m², about 170 g/m², about 180 g/m², about 190 g/m², about 200 g/m², about 210 g/m², about 220 g/m², about 230 g/m², about 240 g/m², about 250 g/m², about 260 g/m², about 270 g/m², about 280 g/m², about 290 g/m², about 300 g/m², about 310 g/m², about 320 g/m², about 330 g/m², about 340 g/m², about 350 g/m², about 360 g/m², about 370 g/m², about 380 g/m², about 390 g/m², about 400 g/m², about 410 g/m², about 420 g/m², about 430 g/m², about 440 g/m², about 450 g/m², about 460 g/m², about 470 g/m², about 480 g/m², about 490 g/m², or about 500 g/m².

The transmission layer may have a basis weight of at least about 100 g/m², about 110 g/m², about 120 g/m², about 130 g/m², about 140 g/m², about 150 g/m², about 160 g/m², about 170 g/m², about 180 g/m², about 190 g/m², about 200 g/m², about 210 g/m², about 220 g/m², about 230 g/m², about 240 g/m², about 250 g/m², about 260 g/m², about 270 g/m², about 280 g/m², about 290 g/m², about 300 g/m², about 310 g/m², about 320 g/m², about 330 g/m², about 340 g/m², about 350 g/m², about 360 g/m², about 370 g/m², about 380 g/m², about 390 g/m², about 400 g/m², about 410 g/m², about 420 g/m², about 430 g/m², about 440 g/m², about 450 g/m², about 460 g/m², about 470 g/m², about 480 g/m², about 490 g/m², or at least about 500 g/m².

The transmission layer may have a basis weight of no more than about 100 g/m², about 110 g/m², about 120 g/m², about 130 g/m², about 140 g/m², about 150 g/m², about 160 g/m², about 170 g/m², about 180 g/m², about 190 g/m², about 200 g/m², about 210 g/m², about 220 g/m², about 230 g/m², about 240 g/m², about 250 g/m², about 260 g/m², about 270 g/m², about 280 g/m², about 290 g/m², about 300 g/m², about 310 g/m², about 320 g/m², about 330 g/m², about 340 g/m², about 350 g/m², about 360 g/m², about 370g/m², about 380 g/m², about 390 g/m², about 400 g/m², about 410 g/m², about 420 g/m², about 430 g/m², about 440 g/m², about 450 g/m², about 460 g/m², about 470 g/m², about 480 g/m², about 490 g/m², or no more than about 500 g/m².

The transmission layer may have a thickness of between about 1.50 and about 6.00 mm, about 1.75 and about 5.75 mm, about 2.00 and about 5.50 mm, about 2.25 and about 5.25 mm, about 2.50 and about 5.00 mm, about 2.75 and about 4.75 mm, about 3.00 and about 4.50 mm, about 3.25 and about 4.25 mm, about 3.50 and about 4.00 mm, or about 3.75 mm.

The transmission layer may have a thickness of about 1.50 mm, about 1.75 mm, about 2.00 mm, about 2.25 mm, about 2.50 mm, about 2.75 mm, about 3.00 mm, about 3.25 mm, about 3.50 mm, about 3.75 mm, about 4.00 mm, about 4.25 mm, about 4.50 mm, about 4.75 mm, about 5.00 mm, about 5.25 mm, about 5.50 mm, about 5.75 mm, or about 6.00 mm.

The transmission layer may have a thickness of at least about 1.50 mm, about 1.75 mm, about 2.00 mm, about 2.25 mm, about 2.50 mm, about 2.75 mm, about 3.00 mm, about 3.25 mm, about 3.50 mm, about 3.75 mm, about 4.00 mm, about 4.25 mm, about 4.50 mm, about 4.75 mm, about 5.00 mm, about 5.25 mm, about 5.50 mm, about 5.75 mm, or at least about 6.00 mm.

The transmission layer may have a thickness of no more than about 1.50 mm, about 1.75 mm, about 2.00 mm, about 2.25 mm, about 2.50 mm, about 2.75 mm, about 3.00 mm, about 3.25 mm, about 3.50 mm, about 3.75 mm, about 4.00 mm, about 4.25 mm, about 4.50 mm, about 4.75 mm, about 5.00 mm, about 5.25 mm, about 5.50 mm, about 5.75 mm, or no more than about 6.00 mm.

Advantageously, a transmission layer as described herein, in particular a transmission layer formed of a 3D spacer fabric as described herein, provides superior airflow, greater compression recovery and superior breathability compared to known transmission layers, in particular transmission layers which are not formed of the described 3D spacer fabric. Thus, the airway of the present invention comprising a transmission layer, in particular a transmission layer formed of a 3D spacer fabric, provides an airway which can be of reduced thickness compared to airways of the prior art while also providing superior airflow, compression recovery and breathability. As such, the present invention provides an airway which can be more comfortable for a patient should the airway be rested or lent upon by the patient, and less likely to cause pressure ulcers and other complications which could otherwise result in significant distress and pain for the patient, while providing superior airflow, compression and breathability compared to airways of the prior art.

In embodiments comprising a plurality of monofilaments separating the upper and lower layers of the transmission layer, monofilaments may be cut, may be removed, or at least partially removed, to facilitate arranging the proximal end of the conduit in the space. Cutting, or removing or partially removing the monofilaments may be performed by any suitable means, including with a scalpel or a stitch unpicker.

The transmission layer may be planar or at least substantially planar. That is to say, the transmission layer may be generally flat, with a substantially greater width and length than its height. Advantageously, this means that the airway can be planar, or at least substantially planar. As such, the present invention provides an airway which is more comfortable for a patient, for example in the event that the patient rests or lies upon the airway when in use, compared to airways of the prior art.

In an embodiment of the invention, there is provided an airway for a pressure gradient wound therapy device comprising: a transmission layer comprising a proximal end, an elongate middle portion and a distal end, the transmission layer comprising an upper layer and a lower layer defining a space therebetween; a top layer constructed from a liquid impermeable material provided over the transmission layer; a bottom layer constructed from a liquid impermeable material provided below the transmission layer, wherein the top layer and the bottom layer enclose at least a portion of the transmission layer; and a conduit comprising a proximal end and a distal end, wherein the proximal end of the conduit is arranged in the space between the upper and lower layers of the proximal end of the transmission layer, wherein the transmission layer is planar, or at least substantially planar.

The transmission layer may have a rectangular, or at least substantially rectangular, cross-section. The transmission layer may have any suitable cross-sectional dimension, for example oval.

The conduit may extend into the proximal end of the transmission layer to a distance equal to about 1%, about 2%, about 3%, about 4%, about 5%, about 6%, about 7%, about 8%, about 9%, about 10%, about 12%, about 15%, about 20%, about 25%, about 30%, about 35%, about 40%, about 45%, about 50%, about 55%, about 60%, about 65%, about 70%, about 75%, about 80%, about 85%, about 90%, or about 95% of the length of the transmission layer.

The conduit may extend into the proximal end of the transmission layer a distance of at least about 1%, about 2%, about 3%, about 4%, about 5%, about 6%, about 7%, about 8%, about 9%, about 10%, about 12%, about 15%, about 20%, about 25%, about 30%, about 35%, about 40%, about 45%, about 50%, about 55%, about 60%, about 65%, about 70%, about 75%, about 80%, about 85%, about 90%, or at least about 95% of the length of the transmission layer.

The conduit may extend into the proximal end of the transmission layer a distance of no more than about 1%, about 2%, about 3%, about 4%, about 5%, about 6%, about 7%, about 8%, about 9%, about 10%, about 12%, about 15%, about 20%, about 25%, about 30%, about 35%, about 40%, about 45%, about 50%, about 55%, about 60%, about 65%, about 70%, about 75%, about 80%, about 85%, about 90%, or no more than about 95% of the length of the transmission layer.

The space between upper and lower layers of the transmission layer may extend the whole length of the transmission layer such that the space forms an elongate channel through the transmission layer. The transmission layer serves to hold the top and bottom (film) layers apart, as such it is desirable that it extends along the whole length of the transmission layer.

The transmission layer may have a thickness between the upper layer and the lower layer of about 0.5 mm, about 1.0 mm, about 1.5 mm, about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8.0 mm, about 8.5 mm, about 9.0 mm, about 9.5 mm, or about 10.0 mm.

The transmission layer may have a thickness between the upper layer and the lower layer of at least about 0.5 mm, about 1.0 mm, about 1.5 mm, about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8.0 mm, about 8.5 mm, about 9.0 mm, about 9.5 mm, or at least about 10.0 mm.

The transmission layer may have a thickness between the upper layer and the lower layer of no more than about 0.5 mm, about 1.0 mm, about 1.5 mm, about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8.0 mm, about 8.5 mm, about 9.0 mm, about 9.5 mm, or no more than about 10.0 mm.

The transmission layer may have a thickness between the upper layer and the lower layer of from about 1.0 mm to about 10.0 mm, from about 1.0 mm to about 9.5 mm, from about 1.0 mm to about 9.0 mm, from about 1.0 mm to about 8.5 mm, from about 1.0 mm to about 8.0 mm, from about 1.0 mm to about 7.5 mm, from about 1.0 mm to about 7.0 mm, from about 1.0 mm to about 6.5 mm, from about 1.0 mm to about 6.0 mm, from about 1.0 mm to about 5.5 mm, from about 1.0 mm to about 5.0 mm, from about 1.0 mm to about 4.5 mm, from about 1.0 mm to about 4.0 mm, from about 1.0 mm to about 3.5 mm, from about 1.0 mm to about 3.0 mm, from about 1.0 mm to about 2.5 mm, from about 1.0 mm to about 2.0 mm, or from about 1.0 mm to about 1.5 mm.

The transmission layer may have a thickness between the upper layer and the lower layer of from about 1.5 mm to about 10.0 mm, from about 1.5 mm to about 9.5 mm, from about 1.5 mm to about 9.0 mm, from about 1.5 mm to about 8.5 mm, from about 1.5 mm to about 8.0 mm, from about 1.5 mm to about 7.5 mm, from about 1.5 mm to about 7.0 mm, from about 1.5 mm to about 6.5 mm, from about 1.5 mm to about 6.0 mm, from about 1.5 mm to about 5.5 mm, from about 1.5 mm to about 5.0 mm, from about 1.5 mm to about 4.5 mm, from about 1.5 mm to about 4.0 mm, from about 1.5 mm to about 3.5 mm, from about 1.5 mm to about 3.0 mm, from about 1.5 mm to about 2.5 mm, or from about 1.5 mm to about 2.0 mm.

The transmission layer may have a thickness between the upper layer and the lower layer of from about 2.0 mm to about 10.0 mm, from about 2.0 mm to about 9.5 mm, from about 2.0 mm to about 9.0 mm, from about 2.0 mm to about 8.5 mm, from about 2.0 mm to about 8.0 mm, from about 2.0 mm to about 7.5 mm, from about 2.0 mm to about 7.0 mm, from about 2.0 mm to about 6.5 mm, from about 2.0 mm to about 6.0 mm, from about 2.0 mm to about 5.5 mm, from about 2.0 mm to about 5.0 mm, from about 2.0 mm to about 4.5 mm, from about 2.0 mm to about 4.0 mm, from about 2.0 mm to about 3.5 mm, from about 2.0 mm to about 3.0 mm, or from about 2.0 mm to about 2.5 mm.

The transmission layer may have a thickness between the upper layer and the lower layer of from about 2.5 mm to about 10.0 mm, from about 2.5 mm to about 9.5 mm, from about 2.5 mm to about 9.0 mm, from about 2.5 mm to about 8.5 mm, from about 2.5 mm to about 8.0 mm, from about 2.5 mm to about 7.5 mm, from about 2.5 mm to about 7.0 mm, from about 2.5 mm to about 6.5 mm, from about 2.5 mm to about 6.0 mm, from about 2.5 mm to about 5.5 mm, from about 2.5 mm to about 5.0 mm, from about 2.5 mm to about 4.5 mm, from about 2.5 mm to about 4.0 mm, from about 2.5 mm to about 3.5 mm, or from about 2.5 mm to about 3.0 mm.

The transmission layer may have a thickness between the upper layer and the lower layer of from about 3.0 mm to about 10.0 mm, from about 3.0 mm to about 9.5 mm, from about 3.0 mm to about 9.0 mm, from about 3.0 mm to about 8.5 mm, from about 3.0 mm to about 8.0 mm, from about 3.0 mm to about 7.5 mm, from about 3.0 mm to about 7.0 mm, from about 3.0 mm to about 6.5 mm, from about 3.0 mm to about 6.0 mm, from about 3.0 mm to about 5.5 mm, from about 3.0 mm to about 5.0 mm, from about 3.0 mm to about 4.5 mm, from about 3.0 mm to about 4.0 mm, or from about 3.0 mm to about 3.5 mm.

The transmission layer may have a thickness between the upper layer and the lower layer of from about 3.5 mm to about 10.0 mm, from about 3.5 mm to about 9.5 mm, from about 3.5 mm to about 9.0 mm, from about 3.5 mm to about 8.5 mm, from about 3.5 mm to about 8.0 mm, from about 3.5 mm to about 7.5 mm, from about 3.5 mm to about 7.0 mm, from about 3.5 mm to about 6.5 mm, from about 3.5 mm to about 6.0 mm, from about 3.5 mm to about 5.5 mm, from about 3.5 mm to about 5.0 mm, from about 3.5 mm to about 4.5 mm, or from about 3.5 mm to about 4.0 mm.

The transmission layer may have a thickness between the upper layer and the lower layer of from about 4.0 mm to about 10.0 mm, from about 4.0 mm to about 9.5 mm, from about 4.0 mm to about 9.0 mm, from about 4.0 mm to about 8.5 mm, from about 4.0 mm to about 8.0 mm, from about 4.0 mm to about 7.5 mm, from about 4.0 mm to about 7.0 mm, from about 4.0 mm to about 6.5 mm, from about 4.0 mm to about 6.0 mm, from about 4.0 mm to about 5.5 mm, from about 4.0 mm to about 5.0 mm, or from about 4.0 mm to about 4.5 mm.

The transmission layer may have a thickness between the upper layer and the lower layer of from about 4.5 mm to about 10.0 mm, from about 4.5 mm to about 9.5 mm, from about 4.5 mm to about 9.0 mm, from about 4.5 mm to about 8.5 mm, from about 4.5 mm to about 8.0 mm, from about 4.5 mm to about 7.5 mm, from about 4.5 mm to about 7.0 mm, from about 4.5 mm to about 6.5 mm, from about 4.5 mm to about 6.0 mm, from about 4.5 mm to about 5.5 mm, or from about 4.5 mm to about 5.0 mm.

The transmission layer may have a thickness between the upper layer and the lower layer of from about 5.0 mm to about 10.0 mm, from about 5.0 mm to about 9.5 mm, from about 5.0 mm to about 9.0 mm, from about 5.0 mm to about 8.5 mm, from about 5.0 mm to about 8.0 mm, from about 5.0 mm to about 7.5 mm, from about 5.0 mm to about 7.0 mm, from about 5.0 mm to about 6.5 mm, from about 5.0 mm to about 6.0 mm, or from about 5.0 mm to about 5.5 mm.

The transmission layer may have a thickness between the upper layer and the lower layer of from about 5.5 mm to about 10.0 mm, from about 5.5 mm to about 9.5 mm, from about 5.5 mm to about 9.0 mm, from about 5.5 mm to about 8.5 mm, from about 5.5 mm to about 8.0 mm, from about 5.5 mm to about 7.5 mm, from about 5.5 mm to about 7.0 mm, from about 5.5 mm to about 6.5 mm, or from about 5.5 mm to about 6.0 mm.

The transmission layer may have a thickness between the upper layer and the lower layer of from about 6.0 mm to about 10.0 mm, from about 6.0 mm to about 9.5 mm, from about 6.0 mm to about 9.0 mm, from about 6.0 mm to about 8.5 mm, from about 6.0 mm to about 8.0 mm, from about 6.0 mm to about 7.5 mm, from about 6.0 mm to about 7.0 mm, or from about 6.0 mm to about 6.5 mm.

The transmission layer may have a thickness between the upper layer and the lower layer of from about 6.5 mm to about 10.0 mm, from about 6.5 mm to about 9.5 mm, from about 6.5 mm to about 9.0 mm, from about 6.5 mm to about 8.5 mm, from about 6.5 mm to about 8.0 mm, from about 6.5 mm to about 7.5 mm, or from about 6.5 mm to about 7.0 mm.

The transmission layer may have a thickness between the upper layer and the lower layer of from about 7.0 mm to about 10.0 mm, from about 7.0 mm to about 9.5 mm, from about 7.0 mm to about 9.0 mm, from about 7.0 mm to about 8.5 mm, from about 7.0 mm to about 8.0 mm, or from about 7.0 mm to about 7.5 mm.

The transmission layer may have a thickness between the upper layer and the lower layer of from about 7.5 mm to about 10.0 mm, from about 7.5 mm to about 9.5 mm, from about 7.5 mm to about 9.0 mm, from about 7.5 mm to about 8.5 mm, or from about 7.5 mm to about 8.0 mm.

The transmission layer may have a thickness between the upper layer and the lower layer of from about 8.0 mm to about 10.0 mm, from about 8.0 mm to about 9.5 mm, from about 8.0 mm to about 9.0 mm, or from about 8.0 mm to about 8.5 mm.

The transmission layer may have a thickness between the upper layer and the lower layer of from about 8.5 mm to about 10.0 mm, from about 8.5 mm to about 9.5 mm, or from about 8.5 mm to about 9.0 mm.

The transmission layer may have a thickness between the upper layer and the lower layer of from about 9.0 mm to about 10.0 mm, or from about 9.0 mm to about 9.5 mm.

The transmission layer may have a thickness between the upper layer and the lower layer of from about 9.5 mm to about 10.0 mm.

In an embodiment of the invention, there is provided an airway for a pressure gradient wound therapy device comprising: a transmission layer comprising a proximal end, an elongate middle portion and a distal end, the transmission layer comprising an upper layer and a lower layer defining a space therebetween; a top layer constructed from a liquid impermeable material provided over the transmission layer; a bottom layer constructed from a liquid impermeable material provided below the transmission layer, wherein the top layer and the bottom layer enclose at least a portion of the transmission layer; and a conduit comprising a proximal end and a distal end, wherein the proximal end of the conduit is arranged in the space between the upper and lower layers of the proximal end of the transmission layer, wherein the transmission layer has a thickness between the upper and lower layers of no more than about 10.0 mm.

The thickness of the transmission layer at the proximal end, the elongate middle portion and the distal end may be equal, or at least substantially equal. Advantageously, this means that the transmission layer is substantially planar which means that the airway is significantly more comfortable for a patient, for example in the event that the patient rests or lies upon the airway when in use, compared to airways of the prior art.

The thickness of the transmission layer at the proximal end may be greater than the thickness of the transmission layer at either or both of the elongate middle portion and the distal end. Beneficially, this may facilitate arranging the proximal end of the conduit in the space between the upper and lower layers of the transmission layer because a thicker proximal end of the transmission layer will provide a greater surface area for a user to guide the proximal end of the conduit toward prior to arranging the conduit in the space. The thickness of the transmission layer at the proximal end may be greater than the thickness of the transmission layer at either or both of the elongate middle portion and the distal end on account of the provision of the airway within the proximal end, which may hold the upper and lower layers apart at the proximal end. For example, where the transmission layer is a 3D spacer fabric, the separation of the upper and lower layers may be determined by the length of the monofilaments (which may be curved), and where these are removed/cut and the proximal end of the conduit is inserted, they may be held further apart.

The width of each of the proximal end, elongate middle portion and distal end of the transmission layer may be equal, or at least substantially equal.

The top layer may have a thickness of at least about 0.05 mm, about 0.1 mm, about 0.15 mm, about 0.2 mm, about 0.25 mm, about 0.3 mm, about 0.35 mm, about 0.4 mm, about 0.45 mm, about 0.5 mm, about 0.55 mm, about 0.6 mm, about 0.65 mm, about 0.7 mm, about 0.75 mm, about 0.8 mm, about 0.85 mm, about 0.9 mm, about 0.95 mm, or at least about 1.0 mm.

The top layer may have a thickness of about 0.05 mm, about 0.1 mm, about 0.15 mm, about 0.2 mm, about 0.25 mm, about 0.3 mm, about 0.35 mm, about 0.4 mm, about 0.45 mm, about 0.5 mm, about 0.55 mm, about 0.6 mm, about 0.65 mm, about 0.7 mm, about 0.75 mm, about 0.8 mm, about 0.85 mm, about 0.9 mm, about 0.95 mm, or about 1.0 mm.

The top layer may have a thickness of no more than about 0.05 mm, about 0.1 mm, about 0.15 mm, about 0.2 mm, about 0.25 mm, about 0.3 mm, about 0.35 mm, about 0.4 mm, about 0.45 mm, about 0.5 mm, about 0.55 mm, about 0.6 mm, about 0.65 mm, about 0.7 mm, about 0.75 mm, about 0.8 mm, about 0.85 mm, about 0.9 mm, about 0.95 mm, or no more than about 1.0 mm.

The bottom layer may have a thickness of at least about 0.05 mm, about 0.1 mm, about 0.15 mm, about 0.2 mm, about 0.25 mm, about 0.3 mm, about 0.35 mm, about 0.4 mm, about 0.45 mm, about 0.5 mm, about 0.55 mm, about 0.6 mm, about 0.65 mm, about 0.7 mm, about 0.75 mm, about 0.8 mm, about 0.85 mm, about 0.9 mm, about 0.95 mm, or at least about 1.0 mm.

The bottom layer may have a thickness of about 0.05 mm, about 0.1 mm, about 0.15 mm, about 0.2 mm, about 0.25 mm, about 0.3 mm, about 0.35 mm, about 0.4 mm, about 0.45 mm, about 0.5 mm, about 0.55 mm, about 0.6 mm, about 0.65 mm, about 0.7 mm, about 0.75 mm, about 0.8 mm, about 0.85 mm, about 0.9 mm, about 0.95 mm, or about 1.0 mm.

The bottom layer may have a thickness of no more than about 0.05 mm, about 0.1 mm, about 0.15 mm, about 0.2 mm, about 0.25 mm, about 0.3 mm, about 0.35 mm, about 0.4 mm, about 0.45 mm, about 0.5 mm, about 0.55 mm, about 0.6 mm, about 0.65 mm, about 0.7 mm, about 0.75 mm, about 0.8 mm, about 0.85 mm, about 0.9 mm, about 0.95 mm, or no more than about 1.0 mm.

The transmission layer may be adhered to at least one of the top and bottom layers. The transmission layer may be adhered to at least one of the top and bottom layers by an adhesive, a weld, such as a heat weld, heat lamination, pressure lamination, or heat and pressure lamination.

The top layer may be sealed to the bottom layer, e.g. at their peripheral edges, to form an elongate channel holding the transmission layer therein. In this arrangement, at least a portion of the conduit may also be held within the elongate channel, extending through the seal.

The proximal end of the conduit may be arranged parallel, or at least substantially parallel, to the longitudinal plane of the transmission layer.

The airway may be suitable and intended for use with a portless negative pressure wound dressing or pressure gradient wound therapy device. Advantageously, this means that the negative pressure wound dressing or pressure gradient wound therapy device does not require the components necessary to support a port or the port itself. Such components are typically rigid and can be uncomfortable for a user of the negative pressure wound dressing or pressure gradient wound therapy device if they were to rest upon the wound dressing or therapy device when in use.

By ‘portless’, we mean a negative pressure wound dressing, or pressure gradient wound therapy device, which does not comprise a port. In the absence of an airway, a port is used to connect a source of negative pressure (e.g. via medical tubing) to a wound dressing.

According to a second aspect of the invention, there is provided a negative pressure wound dressing comprising the airway according to the first aspect.

The negative pressure wound dressing may comprise a backing layer, and a wound contact layer, and the airway may be connected to the backing layer. The backing layer may be provided with an aperture, and the aperture may be arranged in fluid communication with the airway. The wound dressing may comprise one or more intervening layers between the wound contact layer and the backing layer, for example one or more of an absorbent (e.g. superabsorbent) layer and a pressure distribution layer between the backing layer and the wound contact layer.

According to a third aspect of the invention, there is provided a method for manufacturing an airway for a pressure gradient wound therapy device, the method comprising:
a. Providing a transmission layer comprising an upper layer and a lower layer separated by a plurality of monofilaments;
b. Cutting a number of the monofilaments to form a space between the upper and lower layers;
c. Inserting at least a portion of a conduit into the space; and
d. Enclosing at least a portion of the transmission layer between a top layer and a bottom layer.

The transmission layer may comprise a proximal end, an elongate middle portion and a distal end.

The space may be formed between the upper and lower layers of the proximal end of the transmission layer.

The top layer may be constructed from a liquid impermeable material provided over the transmission layer.

The bottom layer may be constructed from a liquid impermeable material provided over the transmission layer.

The top layer may be sealed to the bottom layer, e.g. at their peripheral edges, to form an elongate channel holding the transmission layer therein.

The space may extend in to the transmission layer to a distance equal to at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or at least about 95%, of the length of the transmission layer.

The space may extend in to the transmission layer to a distance equal to no more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or no more than about 95%, of the length of the transmission layer.

The space may extend in to the transmission layer to a distance equal to about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, of the length of the transmission layer.

In step b, cutting may include removing or partially removing a number of the monofilaments.

Cutting, or removing or partially removing, the monofilaments may be performed by any suitable means, including with a scalpel or stitch unpicker.

The method may further comprise adhering at least a portion of the conduit to the transmission layer by an adhesive, a weld, such as a heat weld, heat lamination, pressure lamination, or heat and pressure lamination.

The top layer may be adhered to the bottom layer around its periphery to enclose the at least a portion of the transmission layer.

The top layer may be adhered to the bottom layer by an adhesive, a weld, such as a heat weld, heat lamination, pressure lamination, or heat and pressure lamination.

According to a fourth aspect of the invention, there is provided a method for the treatment of a wound, comprising:
a. Providing a wound dressing with an airway comprising:
   i. a transmission layer; and
   ii. a conduit:
wherein the transmission layer comprises an upper layer and a lower layer and defining a space therebetween; and
the conduit comprises a proximal end and a distal end, wherein the proximal end of the conduit is arranged in the space between the upper and lower layers of the transmission layer,
b. Providing a source of negative pressure;
c. Attaching the conduit to the source of negative pressure;
d. Positioning the wound dressing over a wound site to form a sealed cavity over the wound site;
e. Applying negative pressure to the wound site.

The method may be carried out in the order a-e outlined above, or a different order.

The method may comprise applying negative pressure from a pump through a connector connected to the distal end of the conduit.

The connector may be a fluidic connector. The connector may comprise a filter.

The negative pressure may be transmitted through the transmission layer of the airway to the wound site.

The wound dressing may be portless, i.e., the wound dressing may not comprise a port.

The invention according to any of the first to the fourth aspects may of course individually include any one or more of the features, optional or otherwise, of one another.

### DETAILED DESCRIPTION OF THE INVENTION

In order that the invention may be more clearly understood one or more embodiments thereof will now be described, by way of example only, with reference to the accompanying drawings, of which:

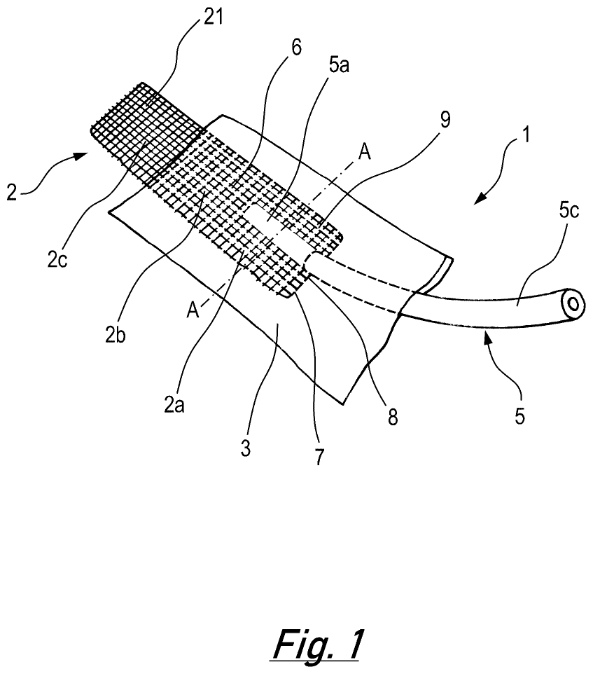

FIG. 1 is a perspective view of a part of an airway according to a first embodiment, with the top layer and bottom layer present;

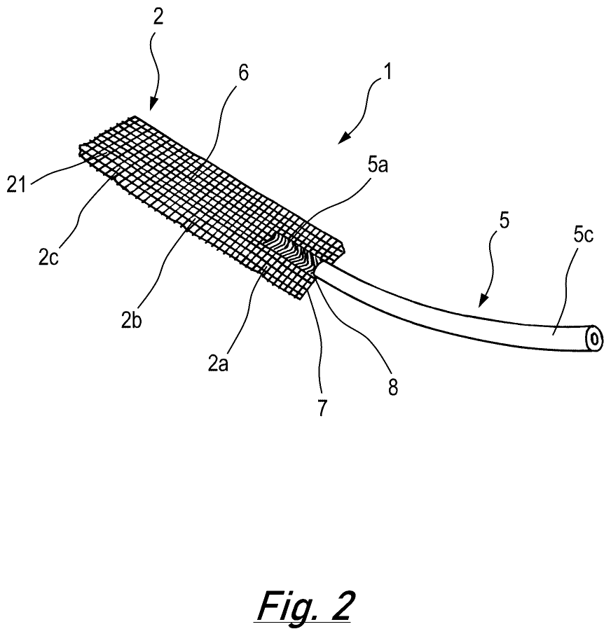

FIG. 2 is a further perspective view of the airway of FIG. 1 with the top and bottom layers not shown;

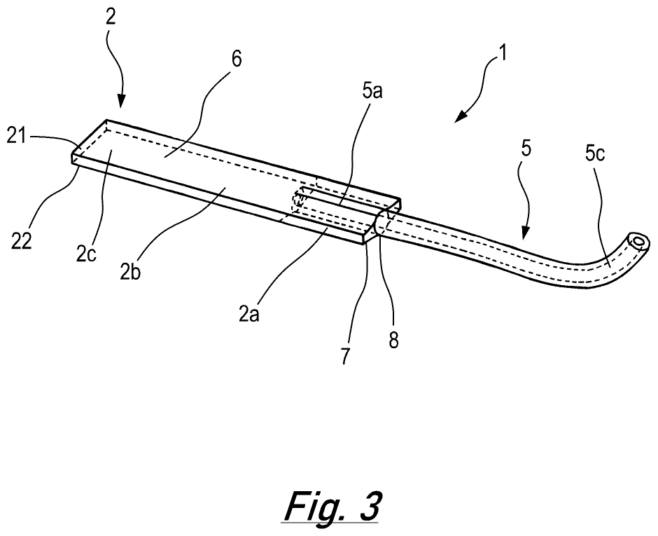

FIG. 3 is a perspective view of the airway with the top layer and bottom layer not shown;

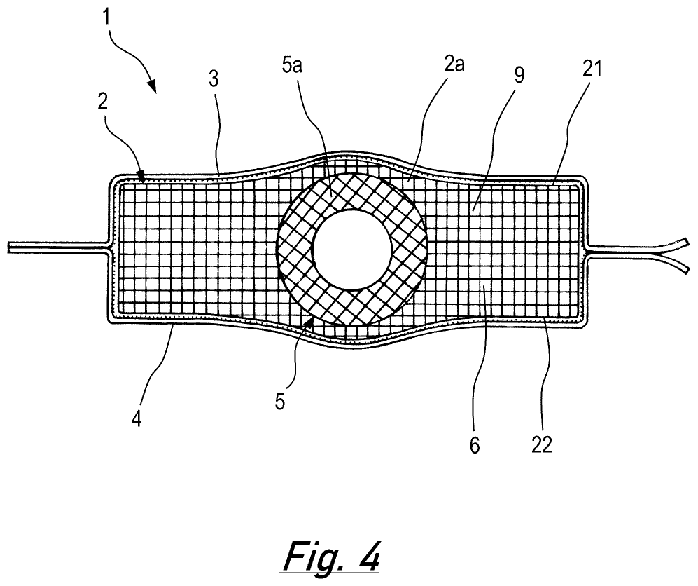

FIG. 4 is a cross section view along line A-A of the part of the airway of FIG. 1; and

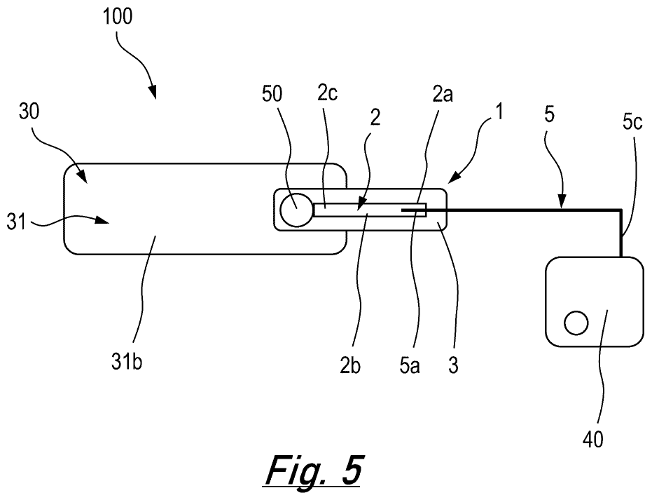

FIG. 5 is a plan view of a negative pressure wound dressing comprising the airway of FIG. 1.

In the Figures, as is conventional, broken lines show hidden features.

With reference to the Figures, the airway 1 comprises a transmission layer 2, a top layer 3, a bottom layer 4 and a conduit 5.

The transmission layer 2 comprises a proximal end 2a, elongate middle portion 2b and a distal end 2c. The transmission layer 2 is elongated and substantially rectangular when viewed in plan view, and the width of each of the proximal end 2a, elongate middle portion 2b and the distal end 2c of the transmission layer 2 are substantially equal. The transmission layer 2 is substantially planar. The transmission layer 2 comprises an upper layer 21 and a lower layer 22, between which is a space 6.

In this embodiment, the conduit 5 is a short section of medical tubing formed of thermoplastic polyurethane and the transmission layer 2 is a 3D spacer fabric. The 3D spacer fabric may be formed of polyester and may be knitted, being formed by warp knitting. The 3D spacer fabric may have a basis weight of between 200 and 250 g/m², preferably 225 g/m². The 3D spacer fabric may have a thickness (i.e., the distance between the upper layer 21 and the lower layer 22) of 2.75 mm.

The space 6 may comprise a plurality of monofilaments separating the upper layer 21 and the lower layer 22. Each monofilament may be joined to an inner surface of the upper layer 21 of the 3D spacer fabric transmission layer 2 and to an inner surface of the lower layer 22 of the 3D spacer fabric transmission layer 2. Each of the plurality of monofilaments may be formed of polyester.

The proximal end 2a comprises an edge 7 which may comprise an aperture 8 which facilitates insertion of a proximal end 5a of the conduit 5 into the space 6. The aperture 8 may be formed by cutting the monofilament at the edge 7 of the proximal end 2a of the transmission layer 2.

The space 6 may extend into the transmission layer 2 a distance equal to 25% of the length of the transmission layer 2.

The conduit 5 may extend into the proximal end 2a of the transmission layer 2 a distance equal to about 20% of the length of the transmission layer 2.

The conduit 5 may for example have an external diameter of 3 mm, and may have an internal diameter of 1.5 mm.

In this embodiment, the outer surface of the proximal end 5a of the conduit 5 comprises an adhesive such that upon arranging the proximal end 5a of the conduit 5 in the space 6 between the upper layer 21 and the lower layer 22 of the transmission layer 2, the conduit 5 is adhered to the inside upper and/or lower surfaces of the transmission layer 2 and to any monofilaments adjacent to the proximal end 5a of the conduit 5 in the proximal end 2a of the transmission layer 2.

The airway 1 may further comprise a top layer 3 constructed from a liquid impermeable material. In this embodiment, the top layer 3 is a polyurethane film. The top layer 3 may be provided over the transmission layer 2 and the proximal end 5a of the conduit 5.

The airway 1 may further comprise a bottom layer 4 constructed from a liquid impermeable material. In this embodiment, the bottom layer 4 is a polyurethane film. The bottom layer 4 is provided below the transmission layer 2 and the proximal end 5a of the conduit 5.

The top layer 3 and the bottom layer 4 may each be waterproof, gas impermeable and moisture vapour permeable.

The top layer 3 and the bottom layer 4 may enclose the transmission layer 2 and the proximal end 5a of the conduit 5.

Each of the top layer 3 and the bottom layer 4 may have a thickness of 0.2 mm.

The top layer 3 may be adhered to the bottom layer 4 around its periphery to form a substantially rectangular elongate channel 9 holding the transmission layer 2 and the proximal end 5a of the conduit 5 therein. The top layer 3 may for example be adhered to the bottom layer 4 by heat lamination.

FIG. 5 shows an example of a negative pressure wound dressing 100 comprising the airway 1. In use as part of the negative pressure wound dressing 100, the conduit 5 may be attached at its distal end 5c to a pump 40 which functions as a source of negative pressure and is capable of supplying negative pressure, via a connector, such as a luer-lock (not shown).

A wound dressing 30 generally includes a covering layer 31 and an adhesive layer (not shown) for adhering the wound dressing adjacent a wound (not shown). In certain embodiments, the wound dressing further comprises a wound contact layer for contacting the wound, a pressure dispersion layer and a plurality of absorbent material layers disposed between the wound contact layer and the pressure dispersion layer.

Generally, the covering layer 31 has a first surface and a second surface 31b, and the first surface is adjacent, and in contact with, the pressure dispersion layer and the adhesive layer. The second surface 31b is an outer facing surface. The covering layer 31 may define a cavity in which the pressure dispersion layer is arranged. The covering layer may comprise an aperture, providing fluid communication between the cavity and an environment external to the dressing. The aperture may be arranged in the optimal position for connection to a source of negative pressure 40.

The top layer 3 of the airway 1 and the bottom layer 4 of the airway 1 may be unequal in length, with the top layer 3 comprising an extended portion extending beyond the bottom layer 4. In such an arrangement, the bottom layer 4 of the airway 1 may extend below the entire transmission layer 2 and be attached to the second surface of the covering layer in the vicinity of the aperture (but not overlying it). The top layer 3 of the airway 1 may extend over the entire transmission layer 2 and the extended portion of the top layer 3 may extend over the point of attachment between the bottom layer 4 and the second surface of the covering layer, and over the aperture in the covering layer. Thus, the extended portion of the top layer 3 comprises edges which overly the second surface of the covering layer. The extended portion may be attached, for example by an adhesive or lamination, about its edges to the second surface of the covering layer in the vicinity of the aperture, such that the edges of the extended portion of the top layer 3 together with the edge of the bottom layer 4 surround the aperture in the covering layer to form a seal. As such, the conduit 5 is in fluid communication with the aperture in the covering layer and, therefore, with the wound site. The seal is preferably air-tight, or at least substantially air-tight, to allow for the efficient supply of negative pressure to the wound dressing to aid the wound-healing process. It will be appreciated by those skilled in the art that a completely air-tight seal is ideal, but with a sufficiently powerful pump, the seal need not be absolutely air-tight.

Alternatively, the top layer 3 may be arranged over the entire transmission layer 2 and the bottom layer 4 may be arranged below the entire transmission layer 2. The top layer 3 and the bottom layer 4 may be sealed together around their periphery, except for where the proximal end 5a of the conduit 5 is positioned between the top layer 3 and the bottom layer 4. The bottom layer 4 may comprise an opening 50 which is positioned in fluid communication with the aperture on the second surface 31b of the covering layer 31. The bottom layer 4 may be attached to the second surface 31b of the covering layer 31, for example by an adhesive or lamination, around the periphery of the aperture on the covering layer or around the periphery of the bottom layer 4, so as to form a seal between the bottom layer 4 and the second surface 31b of the covering layer 31, around the opening 50 and the aperture.

Upon activation of the pump 40, air is drawn toward the pump 40 from the wound site, through the wound dressing 30 and the elongate channel 9 and along the conduit 5, and a negative pressure is therefore transmitted to the wound site.

The arrangement of the proximal end 5a of the conduit 5 in the space 6 between the upper 21 and lower 22 layers of the proximal end 2a of the transmission layer 2 advantageously significantly reduces the probability of the conduit 5 piercing the top layer 3 and/or the bottom layer 4 in use. This is because the proximal end 5a of the conduit 5 is arranged in the space 6 between the upper layer 21 and the lower layer 22 of the transmission layer 2. As such, the 3D spacer fabric of the transmission layer 2, and in particular the upper layer 21 and the lower layer 22, acts as a physical barrier between the proximal end 5a of the conduit 5 and the top layer 3 and bottom layer 4 to substantially reduce the probability of the conduit 5 piercing the film layers 3, 4. Thus, the airway 1 may be more reliable in transmitting a negative pressure to a wound site compared to airways of the prior art.

Further, the airway 1 is of reduced thickness compared to airways of the prior art. At its thickest part, the thickness of the airway 1 is equal to the thickness of the conduit 5, the top and bottom layers 3, 4 and the transmission layer 2. In comparison to the prior art, the transmission layer 2 is not required to be folded or otherwise manipulated to accommodate the conduit 5. As such, the airway 1 may be more comfortable for a patient should the airway 1 be rested or lent upon by the patient. Moreover, the airway 1 is therefore less likely to cause pressure ulcers and other complications which could otherwise result in significant distress and pain for the patient.

Moreover, the airway 1 is simpler to manufacture compared to airways of the prior art. Comparatively, the airway 1 is simple to prepare ready for use in that all that is required is the proximal end 5a of the conduit 5 to be arranged in the space 6 between the upper layer 21 and the lower layer 2 of the transmission layer 2. In the described embodiment which comprises a plurality of monofilaments between the upper layer 21 and the lower layer 22, these filaments are easily cut prior to insertion of the proximal end 5a of the conduit 5 in the space 6. As such, the present invention provides an airway 1 which is easy to prepare ready for use, and which offers little to no risk of damaging the components of the airway 1 in its preparation.

The one or more embodiments are described above by way of example only. Many variations are possible without departing from the scope of protection afforded by the appended claims.

1. An airway for a pressure gradient wound therapy device comprising:
a transmission layer comprising a proximal end, an elongate middle portion and a distal end, the transmission layer comprising an upper layer and a lower layer defining a space therebetween;
a top layer constructed from a liquid impermeable material provided over the transmission layer;
a bottom layer constructed from a liquid impermeable material provided below the transmission layer, wherein the top layer and the bottom layer enclose at least a portion of the transmission layer; and
a conduit comprising a proximal end and a distal end, wherein the proximal end of the conduit is arranged in the space between the upper and lower layers of the proximal end of the transmission layer.

2. An airway according to claim 1, wherein the transmission layer is a foam, a 3D spacer fabric, a 3D knitted material, a porous material or a non-woven material.

3. An airway according to claim 2, wherein the 3D spacer fabric is formed of polyester.

4. An airway according to claim 2, wherein the foam is a polyurethane foam.

5. An airway according to any preceding claim, wherein the transmission layer is a 3D spacer fabric comprising a plurality of monofilaments separating the upper and lower layers of the transmission layer.

6. An airway according to any preceding claim, wherein the transmission layer is planar, or at least substantially planar.

7. An airway according to any preceding claim, wherein the conduit extends into the proximal end of the transmission layer no more than 20% of the length of the spacer layer.

8. An airway according to any preceding claim, wherein the space extends in to the transmission layer to a distance equal to at least 20% of the length of the transmission layer.

9. An airway according to any preceding claim, wherein the transmission layer has a thickness between the upper and lower layers of no more than about 10.0 mm.

10. An airway according to any preceding claim, wherein the width of each of the proximal end, elongate middle portion and distal end of the transmission layer are substantially equal.

11. An airway according to any preceding claim, wherein the transmission layer has a substantially rectangular cross-sectional dimension.

12. An airway according to any preceding claim, wherein the transmission layer is adhered to at least one of the top and bottom layers.

13. An airway according to any preceding claim, wherein the top layer and the bottom layer are formed of polyurethane.

14. An airway according to any preceding claim, wherein the top layer is adhered to the bottom layer to form an elongate channel holding the transmission layer therein.

15. An airway according to any preceding claim, wherein the conduit is medical tubing.

16. An airway according to any preceding claim for use with a ‘portless’ negative pressure wound dressing.

17. A negative pressure wound dressing comprising the airway according to any preceding claim.

18. A method for manufacturing an airway for a pressure gradient wound therapy device, the method comprising:
a. Providing a transmission layer comprising a proximal end, an elongate middle portion and a distal end, the transmission layer comprising an upper layer and a lower layer separated by a plurality of monofilaments;
b. Cutting a number of the monofilaments to form a space between the upper and lower layers of the proximal end of the transmission layer;
c. Inserting at least a portion of a conduit into the space; and
d. Enclosing at least a portion of the transmission layer between a top layer constructed from a liquid impermeable material provided over the transmission layer and a bottom layer constructed from a liquid impermeable material provided below the transmission layer.

19. A method for the treatment of a wound, comprising:
a. Providing a wound dressing with an airway comprising:
i. a transmission layer; and
ii. a conduit:
wherein the transmission layer comprises an upper layer and a lower layer and defining a space therebetween; and
the conduit comprises a proximal end and a distal end, wherein the proximal end of the conduit is arranged in the space between the upper and lower layers of the transmission layer,
b. Providing a source of negative pressure;
c. Attaching the conduit to the source of negative pressure;
d. Positioning the wound dressing over a wound site to form a scaled cavity over the wound site:
e. Applying negative pressure to the wound site.

20. The method according to claim 19, wherein negative pressure is applied from a pump through a connector connected to the distal end of the conduit.
